(19) United States
(12) Patent Application Publication
(10) Pub. No.: US 2013/0281772 A1
(43) Pub. Date: Oct. 24, 2013

(54) LAPAROSCOPIC DEVICE INSERTION CANNULA

(71) Applicant: Allergan, Inc., Irvine, CA (US)

(72) Inventors: Pierre Fridez, Froideville (CH); Julien Rion, Morges (CH); Fabien Kagi, Lausanne (CH); Vernon L. Vincent, Santa Barbara, CA (US)

(21) Appl. No.: 13/923,056

(22) Filed: Jun. 20, 2013

Related U.S. Application Data
(63) Continuation of application No. 13/099,249, filed on May 2, 2011, now abandoned.
(60) Provisional application No. 61/343,561, filed on Apr. 30, 2010.

(51) Int. Cl. A61F 5/00

(52) U.S. Cl. CPC A61F 5/0089
USPC 600/37

(57) ABSTRACT

The present invention is directed to a laparoscopic device insertion cannula (“LDIC”). The LDIC can be configured to store a surgical implant and can be formed from a hollow cylindrically shaped tube including a tapered closed tip at a distal end and a cap at a proximal end. The hollow tube can also include an opening adjacent the distal end. The opening can be covered, for example, by a flexible window. The flexible window can include a slit from which the surgical implant can be extracted from the LDIC. The surgical implant can be, for example, a gastric band.

### CROSS REFERENCE TO RELATED APPLICATIONS

This application is a continuation of U.S. patent application Ser. No. 13/099,249, filed May 2, 2011, which claims the benefit and priority of U.S. Provisional Application No. 61/343,561, filed Apr. 30, 2010, each of which are hereby incorporated by reference in their entirety.

### FIELD

The present invention generally relates to a laparoscopic device insertion cannula.

### BACKGROUND

Introducing a surgical device, a gastric band, drains, shunts, catheters, or many other short-term or long-term implantable devices, such as surgical implants, into the abdominal cavity can be accomplished in a variety of ways. Concerns around the various methods include protecting the device from damage, avoidance of contamination of the device, and maintenance of the pneumoperitoneum (CO₂ inflation of the abdominal cavity).

Conventionally, two options exist to insert a surgical implant: insertion via a large trocar cannula; or insertion directly through a pathway a trocar formerly occupied, such as with the trocar removed and the surgical implant passing through the wound with an instrument or band passer. However, laparoscopic surgeons desire to use the smallest trocars possible to reduce the size of the surgical scars and to keep the procedure as minimally invasive as possible. Furthermore, large trocars exist, but are not always available in all hospitals because of their high costs and their rare applications.

Currently, a few manufacturers produce a 15 mm trocar which is large enough for a surgical implant, such as a standard gastric band, to be passed through the inner lumen. However, larger gastric bands will not fit through a 15 mm trocar. Insertion of a surgical device directly through a skin wound has become common with the standard gastric band or simply with a standard laparoscopic instrument. Although damage of standard gastric bands has been rare and implantation can be accommodated with a short learning curve, it may be desirable to further reduce the occurrences of such damage to the gastric bands.

Likewise, concerns about contamination and eventually infection have proven to be mostly theoretical, since infections resulting from this method are essentially unreported. However, it would be desirable for several reasons to avoid direct skin contact/contamination and to provide a means for avoidance of damage of devices during introduction into the abdominal cavity.

### SUMMARY

The present invention is directed to a laparoscopic device insertion cannula (“LDIC”). The LDIC accomplishes both protection of the device and avoidance of possible contamination, and eliminates the need for expensive and large diameter trocars. It also avoids loss of CO₂ gas (pneumoperitoneum) without a valve or cap common to trocars. The LDIC can be configured to store a surgical implant and can be formed from a hollow tube including a tapered closed tip at a distal end and a cap at a proximal end. The hollow tube can also include an opening adjacent the distal end. The opening can be covered, for example, by a flexible window. The flexible window can include a slit from which the surgical implant can be extracted from the LDIC.

In one embodiment, the present invention is a laparoscopic device insertion cannula including a hollow tube including a distal end, a proximal end, and a tapered tip located at the distal end, wherein the hollow tube defines an opening adjacent the distal end and is configured to store a surgical implant.

In another embodiment, the present invention is a surgical implant package including a sterilized package, a laparoscopic device insertion cannula including a hollow tube located in the sterilized package having a distal end, a proximal end, and a tapered tip located at the distal end, wherein the hollow tube defines an opening adjacent the distal end, and a surgical implant located within the hollow tube.

In yet another embodiment, the present invention is a method for implanting a surgical implant in a patient's abdomen including inserting a laparoscopic device insertion cannula into a patient's abdominal wall, and extracting a surgical implant from an opening in the laparoscopic device insertion cannula.

### BRIEF DESCRIPTION OF THE DRAWINGS

FIG. 1 is a perspective view of a laparoscopic device insertion cannula according to an embodiment of the present invention.

FIG. 2 is a side view of a laparoscopic device insertion cannula according to an embodiment of the present invention.

FIG. 3 is a side view of a laparoscopic device insertion cannula according to an embodiment of the present invention.

FIG. 4 is a perspective view of a gastric band being removed from a laparoscopic device insertion cannula according to an embodiment of the present invention.

FIG. 5 is a sterile packaging for a laparoscopic device insertion cannula according to an embodiment of the present invention.

FIG. 6 is a sterile packaging for a laparoscopic device insertion cannula according to an embodiment of the present invention.

FIG. 7 is a perspective view of a laparoscopic device insertion cannula which is removed from the sterile packaging according to an embodiment of the present invention.

FIG. 8 is a perspective view of a gastric band being removed from a laparoscopic device insertion cannula according to an embodiment of the present invention.

FIG. 9 is a process according to an embodiment of the present invention.

### DETAILED DESCRIPTION

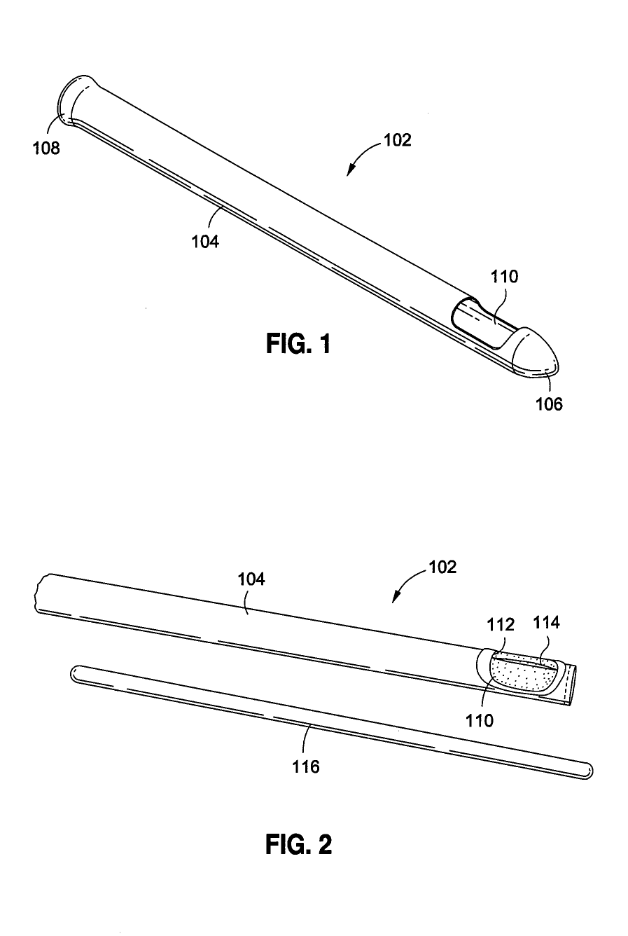

As seen in FIG. 1, the Laparoscopic Device Insertion Cannula (LDIC) 102 is a straight, clear, and rigid cylindrically shaped plastic tube 104 with a conical, tapered closed tip (distal end) 106 for blunt passage through the wound path created by a laparoscopic trocar. A surgical implant can be pre-loaded in the cannula. The back, outside end (proximal end) of the tube is closed with a cap 108 or plug component. In one embodiment, the surgical implant and the complete LDIC 102 is sterilized as one unit. The LDIC 102 has an opening 110 near the distal end, only a short length along the side of the LDIC 102. The opening 110 has a length of between about 5-15 percent of the entire length of the complete LDIC 102. When viewed from above, the opening 110 may be formed in the shape of a rectangle with tapered corners or an oval. The opening 110 is positioned at or near the distal end 106 of the LDIC 102 to allow for easy removal of the surgical implant from the inside of the LDIC 102. The wall of the LDIC 102 is generally rigid and able to be inserted through the skin and facial wounds created by the initial smaller trocar. The LDIC 102 can be made of a clear “see-through” plastic material to allow a surgeon to view the contents (e.g., the surgical implant) inside the cannula.

As seen in FIG. 2, a small area around the opening 110 is a flexible window 112 which is made of a clear material such as silicone, pliable plastic, or flexible plastic so it can expand and allow the implant device to be pulled out of the LDIC 102. The flexible window 112 can include a slit 114 cut lengthwise along the flexible window 112. FIG. 2 also depicts a rod 116 as a comparison for the LDIC 102.

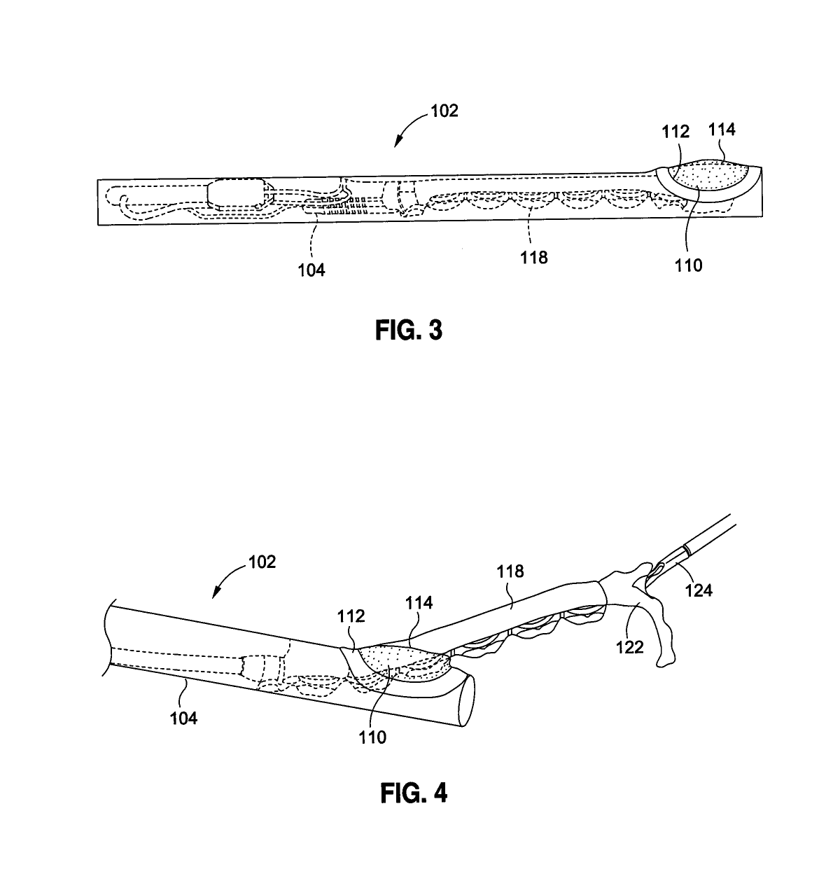

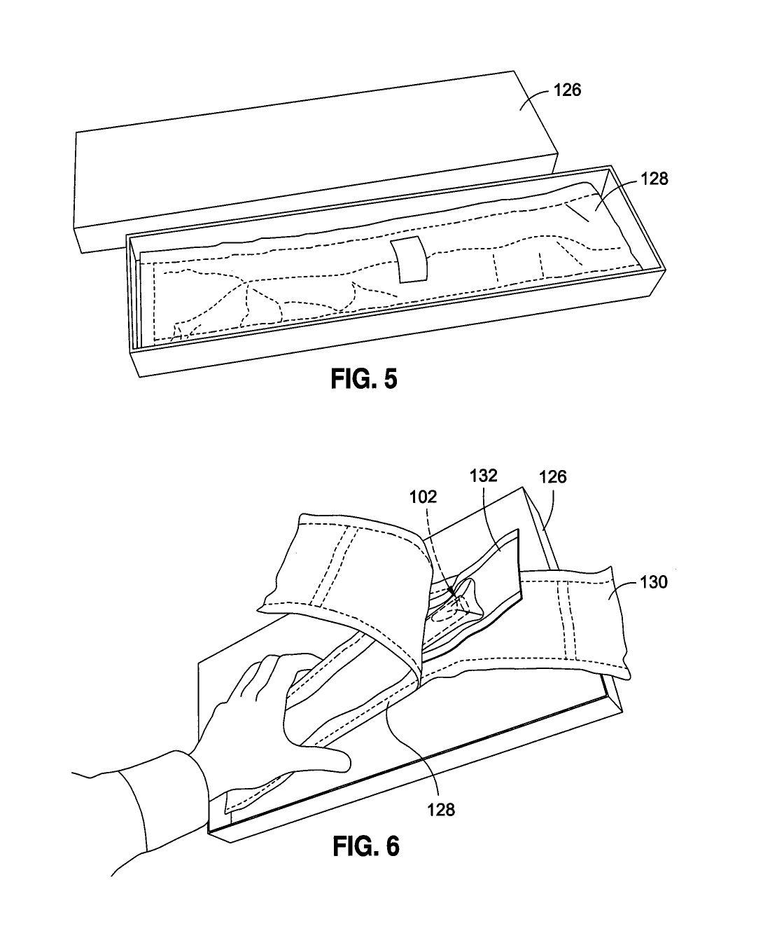

As seen in FIG. 3, the surgical implant 118 can be, for example, a gastric band. The term surgical implant 118 may also be referenced as the gastric band 118. However, the surgical implant is not limited to just being the gastric band. As seen in FIG. 4, with the LDIC 102, a surgeon may manually pull the surgical implant 118 out through the slit 114 in the flexible window 112 that creates the opening 110 at the distal end of the LDIC 102. An individual stylet or obturator may be provided as an accessory to facilitate insertion of the LDIC 102. Use of the LDIC 102 will be as described below.

A surgeon may insert the guiding rod (stylet or obturator) either through the lumen of a standard laparoscopic trocar, or alongside the trocar. The trocar can then be removed from the patient's abdominal wall, leaving the guiding rod in the wound tract created to place the trocar. This maintains the pathway. The LDIC 102 is then passed along side the guiding rod, dilating the original wound tract until the distal tip of the LDIC 102 is inside the abdominal cavity. Surgical lubricant may be necessary to facilitate passage of the LDIC 102.

Surgical expansion of the facial defect may be necessary to allow for the LDIC 102 to pass; a simple extension of the wound in the facia of the rectus muscle with a standard scalpel or by blunt dissection by the surgeon. This may be a common step necessary for other larger non-cutting trocars. This can be done under direct vision by the surgeon, viewing the insertion on a monitor from the laparoscopic camera.

Once the LDIC 102 has been inserted far enough that the open slit 114 on the side of the LDIC 102 is fully inside the abdominal cavity, the surgeon can use a laparoscopic grasper 124 to remove the surgical implant 118 from the LDIC 102. For example, the jaws of the laparoscopic grasper 124 can be inserted through the slit 114 in the flexible window 112 and a buckle tab 122 in the distal end of the surgical implant 118 can be gently grasped. The buckle tab 122 can include, for example, a material that is easily graspable by the laparoscopic grasper 124. The surgical implant 118 is pulled out from the slit 114 in the flexible window 112 until it is completely free in the abdominal cavity.

The LDIC 102 is removed from the wound, leaving the guiding rod in place to maintain the pathway. A standard trocar can then be repositioned, guiding it over, or alongside the guiding rod through the abdominal wall layers. The guiding rod is then removed and the surgery proceeds.

Specific to application with a gastric band or a telemetric adjustable gastric band, the gastric band 118 can be inserted buckle first, into the LDIC 102 so the buckle component is positioned near the introducer conical distal end of the LDIC 102, next to the level of the slit 114 in the flexible window 112. The gastric band tubing or lead and antennae are carefully loaded into the LDIC 102 behind the gastric band component (a manufacturing process). The outer cap 108 or plug is then placed, closing the gastric band 118 inside the LDIC 102.

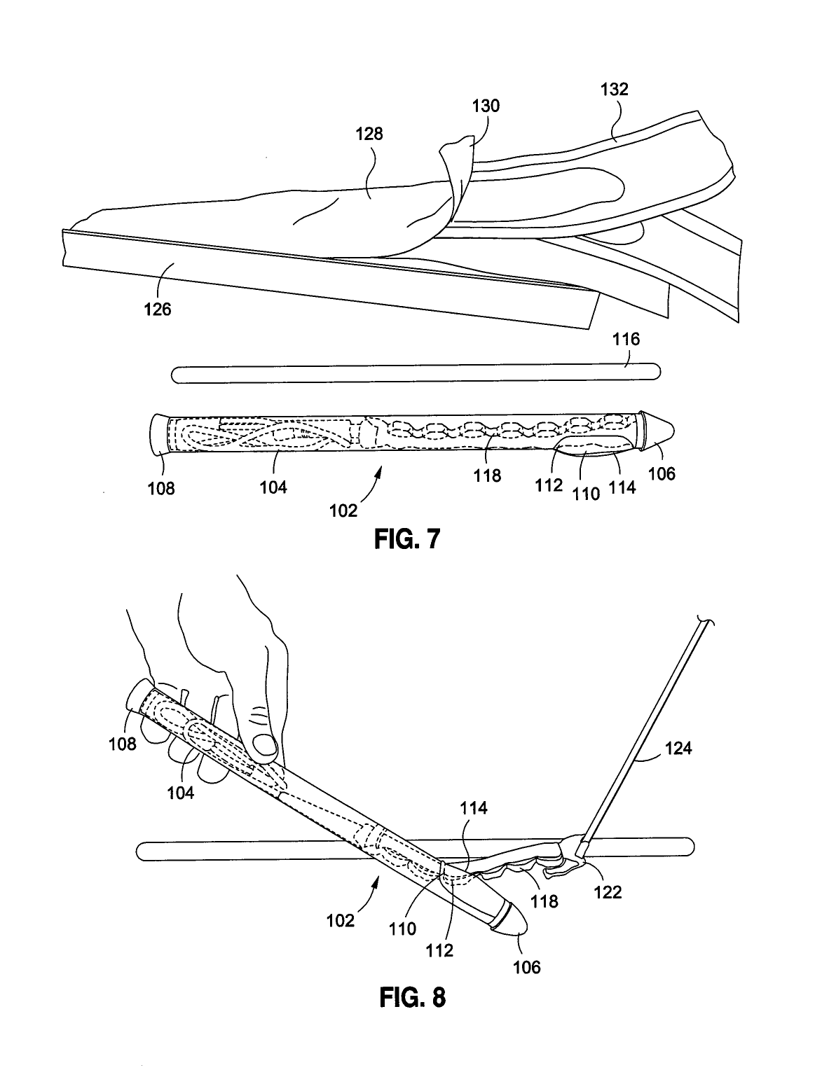

As seen in FIG. 5, the entire assembly can then be packaged (peel pouch, tray, or other standard packaging) and sterilized, for example, in a sterile packaging 128. The entire assembly can form, for example, a surgical implant package. The sterile packaging can be placed, for example, in a box 126. In one embodiment, the sterile packaging can be, for example, a double foil pouch. In the operating room, the LDIC 102 is removed from the sterile packaging 128 and the complete assembly is handed to the surgeon for passage as described above and seen in FIG. 6 and FIG. 7. As seen in FIG. 6, an inner packaging 132 and an outer packaging 130 from the sterile packaging 128 can be opened. In FIG. 7, the LDIC 102 has been removed from the inner packaging 132 and the outer packaging 130, and is ready to be given to the surgeon. The rod 116 may supplement the LDIC 102 and/or optionally be included in the sterile packaging 128. As seen in FIG. 8, the buckle tab 122 can be the optimal component for the surgeon to grasp to remove the gastric band 118 from the LDIC 102, through the slit 114 in the flexible window 112 along the distal end of the LDIC 102.

The gastric band 118 is protected from possible inadvertent contamination by the surgical team in the advent of compromised sterile technique, contaminated glove, or other events which may contaminate the gastric band 118. Possible contamination with skin organisms can be avoided as the gastric band 118 would not come in contact with the patient's skin.

Passage directly through the trocar wound with an introducer instrument can be difficult as the path is not a simple straight line through the tissue layers. Gastric bands can be “lost” off the introducer and need to be blindly retrieved, which has resulted in rare device damage and/or patient injury (local sub-Q trauma). Passing the gastric band 118 via the LDIC 102 provides the protection that is provided via a trocar without the expense or requirement of a larger trocar.

As the LDIC 102 is closed at the outer, proximal end, the passage will be a snug-fit, which will avoid the loss of CO₂ gas used to create/maintain the pneumoperitoneum for the laparoscopic procedure. This saves time and maintains the open space in the inflated abdominal cavity to allow room for the LDIC 102 to be inserted far enough to expose the slit 114 in the flexible window 112 and allow the gastric band 118 to be pulled out. The LDIC 102 is a structure with no valve mechanism or complex parts which should allow for an economical fabrication. The surgeon and hospital will benefit from not needing to purchase expensive and increasingly rare large trocars. Currently, the only trocar that is big enough to pass a large gastric band is an 18 mm trocar. However, such sized trocars are difficult to find and can be expensive.

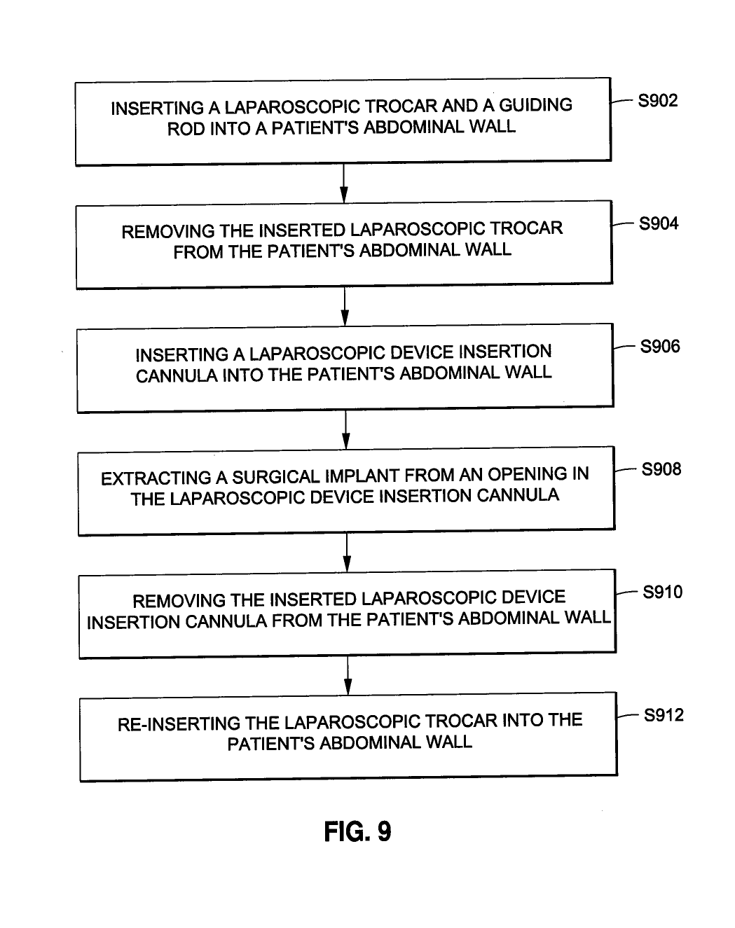

In one embodiment, the present invention is a process as shown in FIG. 9. In Step S902, a laparoscopic trocar and a guiding rod is inserted into a patient's abdominal wall. In Step S904, the laparoscopic trocar is removed from the patient's abdominal wall.

In Step S906, a laparoscopic device insertion cannula is inserted into the patient's abdominal wall. For example, the LDIC 102 shown in FIGS. 1-4 can be inserted into the patient's abdominal wall. In Step S908, a surgical implant is extracted from an opening in the laparoscopic device insertion cannula. For example, the surgical implant 118 can be extracted from the LDIC 102. The surgical implant 118 can be, for example, a gastric band.

In Step S910, the inserted laparoscopic device insertion cannula is removed from the patient's abdominal wall. For example, the LDIC 102 can be removed from the patient's abdominal wall. In Step S912, the laparoscopic trocar is re-inserted into the patient's abdominal wall.

The terms “a,” “an,” “the,” and similar referents used in the context of describing the present invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the present invention otherwise claimed. No language in the specification should be construed as indicating any non-claimed element essential to the practice of the present invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, certain references have been made to patents and printed publications throughout this specification. Each of the above-cited references and printed publications are individually incorporated herein by reference in their entirety.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the present invention disclosed herein are illustrative of the principles of the present invention. Other modifications that may be employed are within the scope of the present invention. Thus, by way of example, but not of limitation, alternative configurations of the present invention may be utilized in accordance with the teachings herein. Accordingly, the present invention is not limited to that precisely as shown and described.

1. A laparoscopic device insertion cannula for holding a gastric band, comprising:
a hollow tube including a distal end, a proximal end, and a tapered tip located at the distal end, the hollow tube defining an opening adjacent to the distal end and configured to hold the gastric band; and
a flexible window covering the opening and having a slit to allow the gastric band to be removed from inside the hollow tube.

2. The device of claim 1 wherein the flexible window is formed from silicone, pliable plastic, or flexible plastic.

3. The device of claim 1 wherein the opening is formed in the shape of a rectangle with tapered corners or an oval.

4. The device of claim 1 wherein the hollow tube includes a cap located at the proximal end.

5. A surgical implant package comprising:
a sterilized package;
a laparoscopic device insertion cannula including
a hollow tube located in the sterilized package having a distal end, a proximal end, and a tapered tip located at the distal end, the hollow tube defining an opening adjacent to the distal end; and
a surgical implant located within the hollow tube.

6. The package of claim 5 wherein the laparoscopic device insertion cannula includes a flexible window covering the opening.

7. The package of claim 6 wherein the flexible window is clear.

8. The package of claim 6 wherein the flexible window is configured to allow the surgical implant to be extracted from the hollow tube.

9. The package of claim 6 wherein the flexible window is formed from at least one of silicone, pliable plastic, or flexible plastic.

10. The package of claim 6 wherein the flexible window defines a slit.

11. The package of claim 5 wherein the surgical implant is a gastric band.

12. The package of claim 5 wherein the hollow tube includes a cap located at the proximal end.

13. A method for implanting a surgical implant in a patient's abdomen comprising:
inserting a laparoscopic device insertion cannula into a patient's abdominal wall; and
extracting a surgical implant from an opening in the laparoscopic device insertion cannula.

14. The method of claim 13 further comprising inserting a trocar and a guiding rod into the patient's abdominal wall prior to inserting the laparoscopic device insertion cannula into the patient's abdominal wall.

15. The method of claim 14 further comprising removing the inserted trocar from the patient's abdominal wall prior to inserting the laparoscopic device insertion cannula into the patient's abdominal wall.

16. The method of claim 15 further comprising:
removing the inserted laparoscopic device insertion cannula from the patient's abdominal wall; and
re-inserting the trocar into the patient's abdominal wall.

17. The method of claim 13 further comprising using a laparoscopic grasper to extract the surgical implant from the laparoscopic device insertion cannula.

18. The method of claim 13 further comprising removing the surgical implant through a slit in a flexible window in the laparoscopic device insertion cannula.

19. The method of claim 13 wherein the surgical implant is a gastric band including a buckle tab.

20. The method of claim 19 further comprising grasping the buckle tab to extract the gastric band from the laparoscopic device insertion cannula.
